(19) United States
(12) Patent Application Publication
(10) Pub. No.: US 2016/0176779 A1
(43) Pub. Date: Jun. 23, 2016

(54) Ethane and Ethylene to Aromatics

(71) Applicant: Pioneer Energy Inc., Lakewood, CO (US)

(72) Inventors: Robert M. Zubrin, Golden, CO (US); Stacy L. Carrera, Golden, CO (US); Mark Berggren, Golden, CO (US)

(73) Assignee: Pioneer Energy Inc., Lakewood, CO (US)

(21) Appl. No.: 14/976,559

(22) Filed: Dec. 21, 2015

Related U.S. Application Data
(60) Provisional application No. 62/096,181, filed on Dec. 23, 2014.

(51) Int. Cl. C07C 2/76; B01J 8/08; B01J 8/02; B01J 8/04; C07C 2/42; C07C 5/333

(52) U.S. Cl. CPC C07C 2/76; C07C 2/42; C07C 5/3332; B01J 8/02; B01J 8/04; B01J 8/0285; B01J 8/08; B01J 8/087; C07C 2529/48; B01J 2208/00389; B01J 2208/00168; B01J 2208/00752

(57) ABSTRACT

This invention pertains to the thermal catalytic synthesis of aromatic compounds from ethane and ethylene. Such synthesis converts lower-value compounds that can only be stored as a gas or liquid under high pressure to a more-valuable liquid compound that can be stored at ambient pressure. The resulting aromatic product is useful as a chemical feedstock or as fuel.

### RELATED APPLICATIONS

This application claims the benefit of U.S. Provisional Application No. 62/096,181 filed Dec. 23, 2014 titled “ETHANE AND ETHYLENE TO AROMATICS” which is incorporated herein by reference in its entirety.

### BACKGROUND OF THE INVENTION

Ethane is an alkane with the chemical formula C₂H₆. It is often present with methane and other alkanes such as propane, butane, hexane, and higher alkanes that are released during oil and natural gas production. Depending on the nature of the oil or gas source, additional compounds such as nitrogen, carbon dioxide, and light hydrocarbons may also be present.

A typical single oil well producing associated gas at a rate of 300,000 standard cubic feet per day (8,490 cubic meters per day) containing 20 percent by volume ethane generates about 5,000 pounds (2,274 kilograms) of ethane daily. There are thousands of such wells in the United States alone.

Ethane can be concentrated and recovered by refrigeration, membrane separations, and other methods resulting in a product largely containing ethane with small amounts of methane or other compounds.

Compared to higher alkanes such as propane or butane, ethane is difficult to store and transport. Ethane can be stored as a liquid at ordinary temperatures at a pressure of about 500 pounds per square inch (psi). Such pressures require special high-strength, thick walls for tanks or pipelines, making transport from oil and gas production sites expensive.

Because ethane is produced in significant quantities and is difficult to transport, its commercial value is low.

In more-remote locations where gas pipelines are not available, ethane and other alkanes produced as associated gas during oil production are often burned in a flare, resulting in carbon dioxide emissions and a loss of income.

If ethane could be converted to a liquid product that is storable at normal ambient pressure, the cost of transporting the product to market could be reduced substantially.

If a more-valuable liquid product could be synthesized from ethane, a substantial profit could be made over that from selling ethane.

### SUMMARY OF THE INVENTION

One novel approach to this major problem is to convert ethane to a transportable, higher-value liquid product is to use a thermal catalytic reactor to synthesize aromatic hydrocarbons. In one example, ethane is converted over a catalyst (such as molybdenum or other metal activated zeolite such as ZSM-5) at temperatures above about 700° C. via the endothermic reaction (1) below to produce benzene and hydrogen.

3C₂H_{6(gas)}=>C₆H_{6(gas)}+6H₂ΔH=+380 kJ at 700° C. (1)

Reaction (1) is favored at lower pressures and can be carried out at pressures near ambient or below. Higher pressures can be used, but equilibrium conversions will be lower.

Benzene and other aromatics produced by this reaction can be condensed and recovered as a liquid product while hydrogen co-produced by the synthesis reaction can be burned to provide heat to sustain the endothermic benzene synthesis reaction.

In addition to the example reaction (1) above, similar reactions can take place to convert ethane to other aromatic compounds including toluene (C₇H₈), xylene (C₈H₁₀) and related compounds containing methyl and ethyl groups as well as C9 and higher aromatic compounds including naphthalene and related compounds.

Refineries routinely operate large-scale thermal (high-temperature) ethane crackers to produce ethylene as feed for plastics or synthesis of other chemicals.

Ethylene can also be used as feed stock for aromatics synthesis.

Another approach to the production of aromatics from ethane is a two-step catalytic conversion process using the same type of catalysts used for synthesis of aromatics from ethane (molybdenum or other metal-activated ZSM-5). Ethane is first converted to ethylene by an endothermic reaction at a temperature above about 800° C., which is in turn converted to aromatics in an exothermic reaction at a temperature above about 400° C. as shown in reactions (2) and (3) below to produce benzene and hydrogen. Reactions (2) plus (3) combined yield the same result as reaction (1) above.

3C₂H_{6(gas)}=>3C₂H_{4(gas)}+3H₂ΔH=+432 kJ at 800° C. (2)

3C₂H_{4(gas)}=>C₆H_{6(gas)}+3H₂ΔH=−57 kJ at 500° C. (3)

In addition to the example reaction (3) above, similar reactions can take place to convert ethylene to other aromatic compounds including toluene (C₇H₈), xylene (C₈H₁₀) and related compounds containing methyl and ethyl groups as well as C9 and higher aromatic compounds including naphthalene and related compounds.

Regardless of whether a one-step or two-step synthesis approach is taken, ethane can be converted to aromatic compounds via thermal catalytic reactions.

The Ethane and Ethylene to Aromatics process is a novel technology to convert alkanes, in particular ethane, to a value-added, storable, transportable, liquid product.

Ethane as feed to the process may be recovered from gas mixtures and may be concentrated prior to conversion to aromatic products.

Alternatively, raw natural gas containing ethane along with methane and higher alkanes can be fed directly to the aromatics synthesis process with increasing yields of aromatics in the order of methane, ethane, propane, butane, pentane, etc.

The hydrogen co-produced with aromatics can be burned, producing only water as a combustion product, which can provide process heat via indirect heat transfer to the catalytic aromatic synthesis reactor.

The Ethane and Ethylene to Aromatics process conditions can be adjusted to tailor the product suite to the desired use. These adjustments can be made to change the relative amounts of lower-carbon aromatics or ethylene produced by the one- or two-step reactions described above.

The Ethane and Ethylene to Aromatics process conditions can be adjusted to minimize coking, or formation of carbon, on reactor or catalyst surfaces which can eventually reduce the yield of desired products and lead to reactor plugging.

The resulting products from the Ethane and Ethylene to Aromatics process can be used directly as fuel or as chemical feedstock.

A gas mixture (such as natural gas) is fed to a one-step thermal catalytic conversion process for synthesis of aromatics either with or without an initial ethane enrichment step (see FIG. 1).

Ethane or an ethane-rich gas is fed to a one-step thermal catalytic conversion process for synthesis of aromatics (see FIG. 1).

A gas mixture (such as natural gas) is fed to a two-step thermal catalytic conversion process first for synthesis of ethylene followed next by synthesis of aromatics either with or without an initial ethane enrichment step (see FIG. 2).

Ethane or an ethane-rich gas is fed to a two-step thermal catalytic conversion process first for synthesis of ethylene followed next by synthesis of aromatics either with or without an initial ethane enrichment step (see FIG. 2).

Although ethylene is not a significant component of natural gas, ethylene produced by the methods described herein or by conventional ethane cracking or other methods may also be used as a feedstock over catalysts similar to those described herein to produce desirable aromatics products.

Various efforts have been conducted to identify economic processes to increase the value of ethane in regions without access to pipelines for ready transport of raw natural gas and natural gas liquids. The present invention is a novel method to synthesize readily transportable, higher-value liquids from ethane that may otherwise be flared or wasted. The present invention includes adjustment of key process conditions such as temperature and flow rate to optimize the overall yield of desirable aromatic liquid product while minimizing detrimental carbon deposition on reactor and catalyst surfaces.

### BRIEF DESCRIPTION OF DRAWINGS

FIG. 1 One-Step Synthesis of Aromatics from Ethane Block Flow Diagram

FIG. 2 Two-Step Synthesis of Aromatics from Ethane Block Flow Diagram

FIG. 3 Experimental Ethane to Aromatics Flow Diagram

FIG. 4 Effect of Temperature on Aromatics Synthesis

FIG. 5 Effect of Ethane Feed Rate on Aromatics Synthesis

FIG. 6 Effect of Time-On-Stream on Aromatics Synthesis

### DETAILED DESCRIPTION OF THE INVENTION

An important aspect of the present invention is a process to convert ethane to aromatic compounds. Ethane may be converted to aromatic compounds using a single-step thermal catalytic process. Alternatively, ethane may be converted to aromatic compounds using a two-step thermal catalytic process in which ethylene is produced as an intermediate product.

The products of the present invention may be used as fuels or blended with fuels or may be used as chemical feedstocks. The products of the present invention may be used as-is or may be further refined to separate individual components.

Various catalysts may be used in reactions of the present application. The catalyst used may contain one or more transition metal such as molybdenum, tungsten, vanadium, Niobium, tantalum, manganese, zirconium, titanium, ruthenium, palladium, platinum, rhodium, nickel, iridium, rhenium, copper, zinc, chromium, nickel, iron, cobalt or combinations thereof. The catalyst may contain a combination of one or more transition metals with main group elements such as for example molybdenum and tin or tungsten and tin. The catalyst may contain promoters such as oxides of barium, magnesium, etc.

Catalysts may be supported or unsupported. A supported catalyst is one in which the active metal or metals are deposited on a support material; e.g. prepared by soaking or wetting the support material with a metal solution, spraying or physical mixing followed by drying, calcination and finally reduction with hydrogen if necessary to produce the active catalyst. Catalyst support materials used frequently are porous solids with high surface areas such as silica, alumina, titania, magnesia, carbon, zirconia, zeolites etc. Zeolites used in the synthesis of aromatics may include ZSM-5, ZSM-22, ZSM-23, SAPO-11, SAPO-41, modernite type, ferrierite, zeolite Y, zeolite beta, zeolite MCM-22, zeolite ZSM-57, zeolite SUZ-4, zeolite EU-1, zeolite SSZ-23, or a mixture of two or more thereof. The zeolites may be synthesized by method described in “Zeolite: Synthesis, Chemistry and Applications” by Andreyev which is incorporated by reference in its entirety herein.

The methods of the present invention can comprise, consist of, or consist essentially of the essential elements and limitations of the method described herein, as well as any additional or optional ingredients, components, or limitations described herein or otherwise useful in synthetic organic chemistry.

Unless specifically noted otherwise herein, the definitions of the terms used are standard definitions used in the art. Exemplary embodiments, aspects and variations are illustrated in the figures and drawings, and it is intended that the embodiments, aspects and variations, and the figures and drawings disclosed herein are to be considered illustrative and not limiting.

In one embodiment aromatic compounds including benzene, toluene, naphthalene, and other C8, C9, and C10 aromatic compounds are synthesized by direct reaction of ethane over a 2% molybdenum-ZSM-5 catalyst or other suitable catalyst.

In one embodiment ethane is present along with other alkanes and hydrocarbons recovered from natural gas wells.

In one embodiment the ethane is concentrated and recovered from natural gas produced from gas wells or as associated gas from oil production.

In one embodiment the ethane is obtained from commercial sources as a feed stock for production of aromatic compounds.

In one embodiment the aromatics synthesis reaction is carried out at a temperature between 700 and 1000° C.

In one embodiment the process heat is supplied by combustion of hydrogen and/or other byproduct gases from ethane decomposition.

In one embodiment the process heat is supplied by electrical heaters.

In one embodiment the aromatics synthesis reaction is carried out at a pressure between 0.1 and 10 bar absolute.

In one embodiment the aromatics synthesis reaction is carried out at a gas hourly space velocity between 50 and 10000/hour.

In one embodiment the catalyst consists of an H-ZSM-5 substrate to which an activating metal or metal oxide such as molybdenum is added.

In one embodiment the catalyst consists of an H-ZSM-5 substrate with an activating compound concentration between 0.05 and 10 percent.

In one embodiment aromatic compounds including benzene, toluene, naphthalene, and other C8, C9, and C10 aromatic compounds are synthesized by a two-step reaction of ethane over a 2% molybdenum-ZSM-5 catalyst or other suitable catalyst first to produce ethylene and hydrogen and next by reaction of the ethylene over a 2% molybdenum-ZSM-5 catalyst or other suitable catalyst to produce aromatic compounds and hydrogen.

In one embodiment the ethane feed gas for a two step aromatics synthesis process is present along with other alkanes and hydrocarbons recovered from natural gas wells.

In one embodiment the ethane feed gas for a two step aromatics synthesis process is concentrated and recovered from natural gas produced from gas wells or as associated gas from oil production.

In one embodiment the ethane feed gas for a two step aromatics synthesis process is obtained from commercial sources as a feed stock for production of aromatic compounds.

In one embodiment a first stage ethane decomposition reaction is carried out at a temperature between 800 and 1000° C.

In one embodiment the first stage ethane decomposition reaction process heat is supplied by combustion of hydrogen and/or other byproduct gases from ethane decomposition.

In one embodiment the first stage ethane decomposition reaction process heat is supplied by electrical heaters.

In one embodiment the first stage ethane decomposition reaction is carried out a pressure between 0.1 and 10 bar absolute.

In one embodiment the first stage ethane decomposition reaction is carried out a gas hourly space velocity between 50 and 10000/hour.

In one embodiment the first stage ethane decomposition reaction catalyst consists of an H-ZSM-5 substrate to which an activating metal or metal oxide such as molybdenum is used.

In one embodiment the first stage ethane decomposition reaction decomposition catalyst consists of an H-ZSM-5 substrate with an activating compound concentration between 0.05 and 10 percent.

In one embodiment the second stage ethylene feed is obtained from the first step of processing over a molybdenum ZSM-5 catalyst or similar catalyst or from conventional cracking of ethane, or from commercial sources as a feed stock for production of aromatic compounds.

In one embodiment the second stage ethylene to aromatics reaction is carried out at a temperature between 300 and 900° C.

In one embodiment the second stage ethylene to aromatics reaction is carried out at a pressure between 0.1 and 10 bar absolute.

In one embodiment the second stage ethylene to aromatics reaction is carried out at gas hourly space velocity between 50 and 10000/hour.

In one embodiment the second stage ethylene to aromatics catalyst consists of an H-ZSM-5 substrate to which an activating metal or metal oxide such as molybdenum is used.

In one embodiment the second stage ethylene to aromatics reaction catalyst consists of an H-ZSM-5 substrate with an activating compound concentration between 0.05 and 10 percent.

In one embodiment a single reactor with different temperature zones is used for the two-step conversion of ethane to aromatics.

In one embodiment two separate reactors are used for the two-step conversion of ethane to aromatics.

In one embodiment non-condensable byproduct gases are separated from any liquid aromatic products prior to feeding a second stage reactor.

In one embodiment hydrogen is separated from any liquid aromatic products and ethane prior to feeding a second stage reactor.

### EXPERIMENTAL

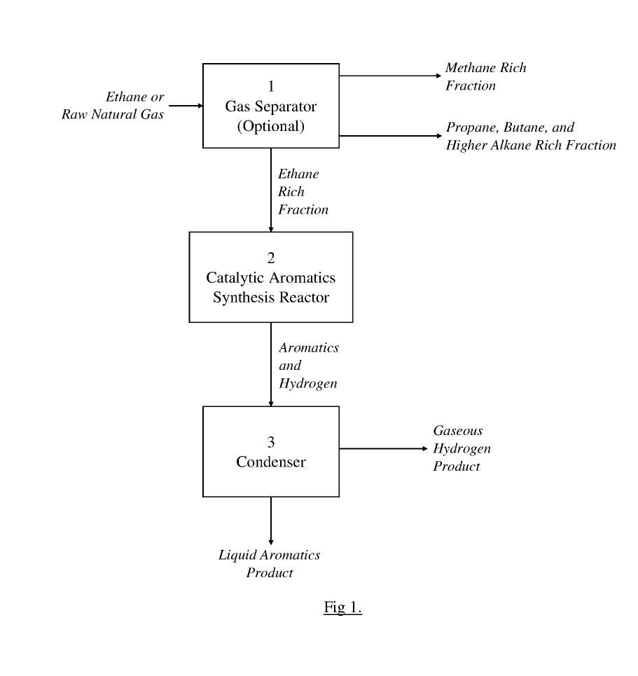

The following procedures may be employed for synthesis of aromatic compounds from ethane. Key hardware for the one-step production of aromatics from ethane consists of an optional ethane concentrating system (based on refrigeration and condensing, membranes, compression, or other means), a thermal catalytic aromatics synthesis reactor, a cooling system, and liquid product condenser (see FIG. 1).

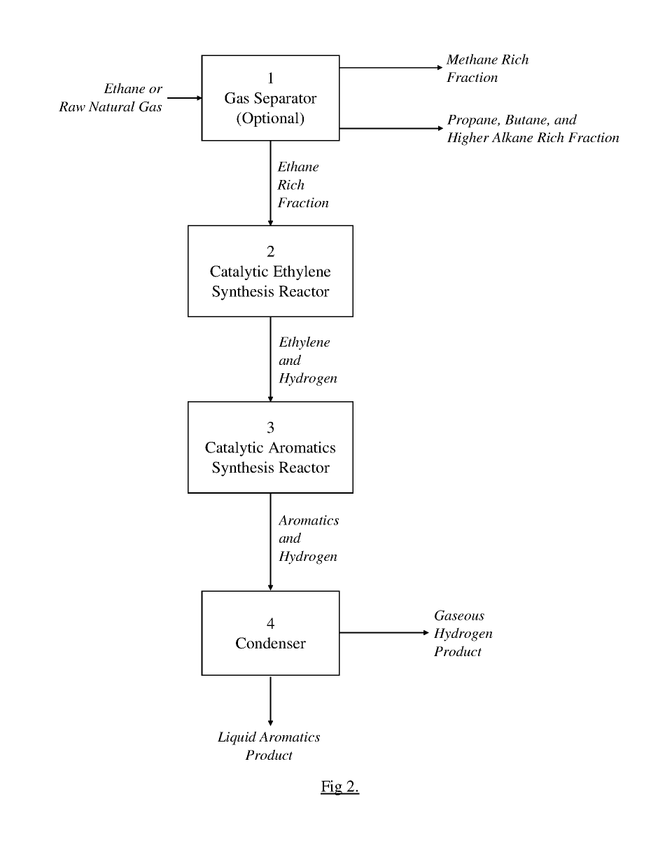

Key hardware for the two-step production of aromatics from ethane consists of an optional ethane concentrating system (based on refrigeration and condensing, membranes, compression, or other means), a thermal catalytic ethane decomposition reactor for production of ethylene gas, a thermal catalytic reactor for synthesis of aromatics from ethylene gas, a cooling system, and liquid product condenser (see FIG. 2).

A reactor of similar design and similar catalyst may be used for the ethane decomposition and aromatics synthesis steps during two-step aromatics synthesis with adjustments to operating conditions for each step such as flow rate and temperature, as required to produce optimal results.

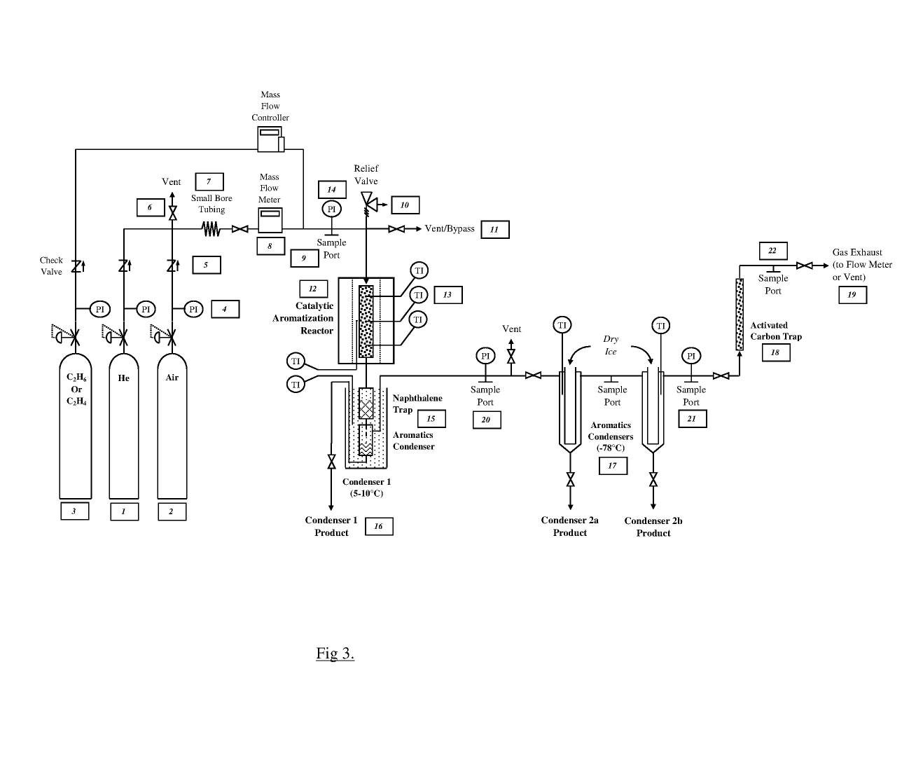

A laboratory apparatus was assembled and placed in a fume hood and was used to conduct experiments described herein (see FIG. 3). Gas cylinders were used to supply feed for experiments. Helium was delivered from a high pressure cylinder 1 through a pressure reducing regulator adjusted on the basis of a pressure gauge 4. Helium was used to purge any oxidizing or reducing gases from the system prior to and after each experiment but was not used during experimentation. A similar arrangement was prepared for delivery of air 2 for catalyst regeneration and for delivery of ethane (C₂H₆) or ethylene (C₂H₄) 3. Ethane cylinders contain liquid ethane with gaseous ethane in the vapor phase at a pressure of about 500 psi. Ethane cylinders were configured for withdrawal of ethane gas present above the liquid. All other cylinders contain pressurized gas of the type indicated.

Feed gases were passed through check valves 5 to prevent back flow. Gases were passed through a mass flow controller (Omega FMA 5416; 0-2 liters per minute based on nitrogen) or mass flow meter (Omega FMA 1712601; 0-500 cubic centimeters per minute based on methane) 8 for adjustment or monitoring of flow. The ethane and ethylene gas supply regulators were set to a pressure of 20 to 25 psig and were passed through the flow controller which was calibrated in advance of testing with each gas to deliver an accurate flow as a function of input control voltage. The helium and air supply gas regulators were set to pressures between about 10 and 100 psi to adjust flow rate based on pressure drop through a 5-inch length of 0.010-inch inside-diameter stainless steel tubing 7. The 0.010-inch inside-diameter tubing was characterized with respect to flow rate versus inlet and outlet pressure for the helium and air gas supplies in advance of testing.

Inlet gas tubing from the individual sources was combined in a tee just upstream of a pressure gauge and sample port 14. A pressure relief valve 10 set to about 30 psi was installed to vent gases automatically in the event of system over-pressurization. A vent/bypass line 11 was installed to allow inlet gases to bypass the reactor if desired.

A down-flow catalytic reactor 12 was used both for one-step synthesis of aromatics from ethane and for synthesis of aromatics from ethylene. The reactor consists of a one-inch outside diameter stainless steel tube with a wall thickness of 0.065 inch and a length of nine inches. Swagelok® compression fittings were used to provide the process and instrumentation connections shown in FIG. 3. A layer of porous ceramic foam was inserted in the bottom fitting of the reactor to provide gas flow across the entire cross section before necking down to the ¼-inch diameter outlet tubing. A layer of ⅛-inch thick alumina felt was placed above the ceramic foam to support the catalyst particles while allowing nearly unrestricted gas flow. Thermocouples 13 were installed through ⅛-inch diameter ports located along the length of the reactor. Additional thermocouples were installed on the reactor shell as needed. A radiant furnace (Omegalux; 2-inch inside diameter; 120 volts, 425 watts) was used to supply heat to the catalytic reactor. Thermocouple readers and a LabJack® data acquisition system were used to monitor and record temperatures.

The exhaust gases from the catalytic reactor were passed through a trap and condenser system 15 consisting of the following components which were submerged in a cold water bath held at a temperature of 5 to 10° C. ¼-inch diameter stainless steel tube directed hot reactor exhaust to a 6-inch length of ¾-inch outside diameter tube with a wall thickness of 0.049 inch. The tubing was loosely packed with coarse stainless steel wool to act as a trap to collect naphthalene which might otherwise cause plugging if present in sufficient quantity. The exhaust from the naphthalene trap was then directed through a ½-inch diameter stainless steel tube to a condensate separator, which consists of a 7-inch length of ¾-inch outside diameter tube with a wall thickness of 0.049 inch. The inlet tubing passes through a tee connection that allows liquid to collect in the separator while non-condensed gases pass overhead to an outlet port connected to ½-inch diameter stainless steel tubing. A length of ⅛-inch diameter tubing is connected to the bottom of the separator to allow for periodic withdrawal of condensed liquid 16 upon application of slight system pressure (about 2 psi) achieved by temporarily closing the system gas exhaust valve.

The pressure of non-condensed gases exiting the condenser system can be read on a pressure gauge and a sample 20 can be taken for analysis. A tee and vent valve are located just down stream of the pressure gauge and sample port to allow bypassing of the down stream condenser system if desired.

The non-condensed gases are directed to a second condenser system 17 which consists of a two-stage freezing condenser cooled by dry ice. Each of two condensers consist of a 2-inch diameter inner tube into which dry ice is placed to act as a cold-finger for freezing and collection of aromatic compounds not collected in the first condenser system 16. The dry-ice containing tube is open at the top and sealed at the bottom and located within a sealed 3-inch outside diameter tube of about 12-inches length fitting with a conical bottom to allow for withdrawal of liquid product after an experiment is completed and the condenser warms to ambient temperature. A baffle is located longitudinally between the external and internal tubes along the cylindrical length to prevent short-circuiting of gases from the inlet to the outlet of each dry-ice condenser vessel. A sample port 21 is located between the two dry-ice condenser vessels and just down stream of the second condenser along with a pressure gauge.

The exhaust gases from the second dry-ice condenser are directed through a ½-inch diameter tube to an activated carbon trap to recover any small amounts of aromatics or other condensable gases not trapped in upstream condensers. The carbon trap consists of an 8-inch length of ¾-inch outside diameter stainless steel tubing of 0.035-inch wall thickness. The trap is filled with about 16 grams of Norit GF45 activated carbon pellets of approximately ⅙-inch diameter.

A sample port 22 is located down stream of the carbon trap, just upstream of the final system exhaust, which is periodically connected to a bubble meter for measurement of the dry exhaust gas flow rate.

The same catalyst was used for single-step synthesis of aromatics from ethane as well as two-step ethane decomposition and aromatics synthesis from ethylene.

The catalyst was prepared with a loading of two weight percent molybdenum as described herein. H-ZSM-5 catalyst substrate (Tricat T-2S; 1.8 millimeter diameter extrudates; SiO₂₁Al₂O₃ ratio=23.5) was used for the experiments. The molybdenum (Mo) was obtained in the form of ammonium heptamolybdate tetrahydrate (Sigma-Aldrich A7302; Formula Weight 1235.86 g/mol). The incipient wetness technique was used to infuse the molybdenum compound into the catalyst substrate. It was first determined that 68 milliliters of water would soak into 150 grams of dry catalyst substrate so that the interior pores were filled and surfaces were moist, but no free water was present. Next, 5.63 grams of ammonium heptamolybdate tetrahydrate (containing 3.0 grams of molybdenum) was dissolved in a fresh sample of 68 milliliters of distilled water which was then transferred to a ceramic dish. A fresh, dry 150 gram sample of H-ZSM-5 catalyst substrate was then added to the ceramic dish containing the molybdenum solution and was gently mixed until the solution was uniformly absorbed into the catalyst.

The catalyst was first dried at about 120° C. in air for about six hours. The dried catalyst containing molybdenum was the calcined at 500° C. in air for about 16 hours and then cooled to ambient temperature.

The reactor described above was loaded with 30.9 grams of the Mo-ZSM-5 catalyst prepared as described above. The height of the 30.9 gram catalyst bed in the one-inch outside diameter reactor was 6.5 inches, resulting in a volume of 63 milliliters. The bulk density as loaded was about 0.5 gram per milliliter.

A set of single-step aromatics synthesis experiments was conducted to identify effects of temperature, flow rate, and time-on-stream on ethane conversion and aromatics yield. Prior to starting each experiment, the Condenser 1 water bath (see FIG. 3) was cooled to about 5° C. with ice and was held in the range of 5 to 10° C. throughout each experiment. The Condenser 2a and 2b cold fingers were loaded with crushed dry ice to pre-chill the condensers. Helium gas was flowed through the system at a rate of 50 to 100 standard cubic centimeters per minute (sccm) to purge air or other gases from the system. The synthesis reactor heater was started and the reactor was heated to the desired reactor shell target temperature. Helium flow was stopped and ethane gas was started at the target flow rate. The heater controls were adjusted as needed to hold temperatures steady during testing. Samples were routinely taken to measure exhaust gas flow (by diverting the vent gas 19 in FIG. 3 to a bubble meter) and exhaust gas composition (using a 4-channel Varian® CP4900 MicroGC controlled with Galaxie® software). The MicroGC was configured to quantitatively measure the volume percentage of hydrogen, oxygen, nitrogen, carbon monoxide, methane, carbon dioxide, ethane, ethylene, propane, propylene, and butane. Periodic gas samples were also taken for semi-quantitative analysis of a wide range of gas constituents using a GC/Mass Spectrometer (Hewlett Packard® HP 6890A gas chromatograph system with an Agilent Technologies® 5973 Network Mass Selective Detector). Condenser samples were collected and retained in the condenser vessels until a run was completed. After each run was completed, the ethane feed gas was stopped, helium was started, and heaters were turned off. After purging gases from the entire system with helium, the Condenser 2a and 2b system valves were closed to isolate the condensers as they warmed overnight. Helium flow was continued to build a pressure of 5 to 10 psi in the feed system, reactor, and Condenser 1. The helium gas pressure was used to withdraw condensate from Condenser 1. The condensate was weighed and stored in glass containers. After warming to ambient temperature overnight, gas pressure built to the range of 2 to 30 psi in the Condenser 2a and 2b system, which allowed for withdrawal of condensate from each vessel. Condensate 2a and 2b samples were weighed and stored in glass containers. All liquid condensate samples were subjected to quantitative analysis by chromatography (GowMac 580 with a 9-foot, ⅛-inch diameter Hayesep Q® separation column and a thermal conductivity detector) for the volume percentage of liquid in the condensate for benzene and toluene. Periodic semi-quantitative analyses were also conducted using the GC/Mass Spectrometer described above. The gas feed rate, gas exhaust rate and composition, and average condensate rates and compositions were used to calculate conversion of ethane as well as yield of aromatics product based on the weight collected in each condenser.

### Experiments 1-10

The experimental system was prepared and pre-heated as described above to a target shell temperature of about 853° C. (internal temperature of about 880° C.). For Experiment 1, the ethane feed gas was then started at a flow of about 103 sccm with the primary goal of converting the molybdenum oxide constituent in the catalyst to the active carbide form. The system was operated for 120 minutes. Subsequent experiments in the series were conducted to determine effects of temperature, flow rate, and time-on-stream on ethane conversion, aromatics yield, ethylene yield, and methane yield. The middle reactor temperature shown in Table 1 was typically the highest temperature obtained in the reactor. Operating conditions and results of Experiments 1 through 10 are summarized in Table 1. The data shown in Table 1 were taken near the end of each test segment.

The effect of ethane feed rate was determined from the results of Experiments 5, 8, 9, and 10 (all from 874 to 890° C. middle reactor temperature at ethane feed rates between 98 and 452 sccm). Results showed that ethane conversion dropped from 93 percent at the lowest ethane feed rate to 83 percent at the highest ethane feed rate. The ethylene yield increased from 39 percent at the lowest ethane feed rate to 70 percent at the highest ethane feed rate. The measured liquid aromatics yield dropped from 15 percent at the lowest ethane feed rate to 6 percent average over all higher ethane feed rates. The combined aromatics plus ethylene yield increased from about 54 percent at the lowest ethane feed rate to about 70 percent at the highest ethane feed rate. This is significant because as discussed later in this application, ethylene may ultimately be converted to aromatic products.

TABLE 1

| | Experiment Number | | | | | | | | | |
|---|---|---|---|---|---|---|---|---|---|---|
| | 1 | 2 | 3 | 4 | 5 | 6 | 7 | 8 | 9 | 10 |
| Time on Stream (minutes) | 120 | 240 | 360 | 480 | 600 | 695 | 750 | 810 | 865 | 905 |
| sccm total gas feed | 110 | 108 | 112 | 111 | 109 | 114 | 230 | 238 | 358 | 487 |
| sccm ethane in gas feed | 103 | 102 | 106 | 102 | 98 | 107 | 216 | 221 | 332 | 452 |
| g/min C in feed ethane | 0.110 | 0.109 | 0.114 | 0.109 | 0.105 | 0.115 | 0.231 | 0.237 | 0.355 | 0.484 |
| total g C fed during segment | 13.2 | 13.1 | 13.6 | 13.1 | 12.6 | 10.9 | 12.7 | 14.2 | 19.6 | 19.4 |
| Reactor Shell Temperature, C. | 853 | 854 | 849 | 847 | 848 | 710 | 710 | 845 | 850 | 845 |
| Reactor Middle Temperature, C. | 880 | 881 | 884 | 891 | 890 | 752 | 752 | 885 | 883 | 874 |
| sccm Exhaust Gas | 185 | 179 | 190 | 183 | 182 | 132 | 286 | 429 | 655 | 867 |
| Exhaust Gas % H2 | 45.5 | 55.6 | 53.4 | 52.0 | 51.0 | 23.0 | 15.7 | 48.9 | 46.5 | 44.6 |
| Exhaust Gas % CH4 | 50.7 | 26.8 | 24.5 | 22.4 | 23.0 | 4.2 | 3.3 | 15.7 | 12.3 | 9.7 |
| Exhaust Gas % C2H4 | 0.0 | 8.9 | 15.2 | 19.4 | 21.6 | 17.7 | 14.4 | 31.7 | 35.0 | 38.0 |
| Exhaust Gas % C2H6 | 0.8 | 7.1 | 5.2 | 4.6 | 3.9 | 57.2 | 69.2 | 4.3 | 5.4 | 9.1 |
| % C2H6 Conversion | 99 | 88 | 91 | 92 | 93 | 29 | 8 | 91 | 90 | 83 |
| % Average Measured | 3 | 24 | 23 | 20 | 15 | 7 | | 6 | | |
| Liquid Yield | | | | | | | | | | |
| % Ethane Conversion to Ethylene | 0 | 16 | 26 | 34 | 39 | 21 | 18 | 59 | 66 | 70 |
| (percent of ethane carbons to ethylene) | | | | | | | | | | |
| % Ethane Conversion to Methane | 46 | 23 | 21 | 20 | 21 | 3 | 2 | 15 | 11 | 9 |

During test 1 (catalyst activation) at an ethane feed rate of 103 sccm and about 880° C. middle reactor temperature, results showed nearly complete ethane conversion (99%), no ethylene production, and only 3 percent yield of liquid aromatics product.

The effect of temperature was established using results from Experiments 2 through 5 (98 to 106 sccm ethane and 880 to 890° C. reactor middle temperature) and Experiments 6 and 7 (107 and 216 sccm ethane and 752° C. reactor middle temperature. The results showed that the average measured liquid aromatics yield was 7 percent by weight of carbon in the feed (reported on a 100 percent benzene product basis) at a middle reactor temperature of 752° C. The measured liquid aromatics yield increased to the 15 to 24 percent range with reactor middle temperatures in the 880 to 890° C. range.

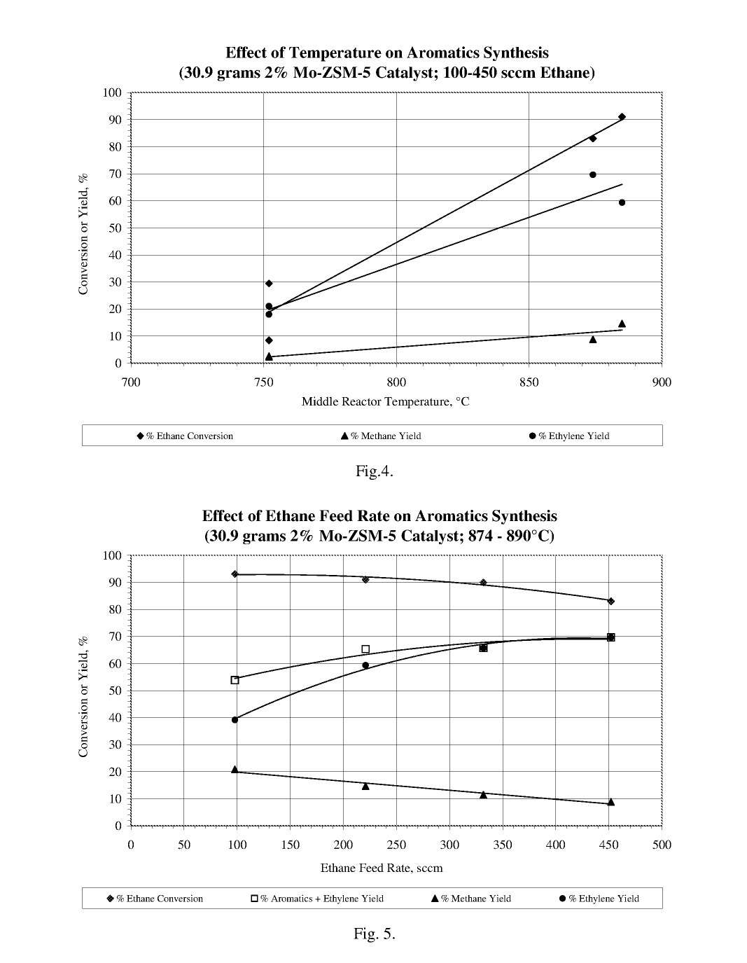

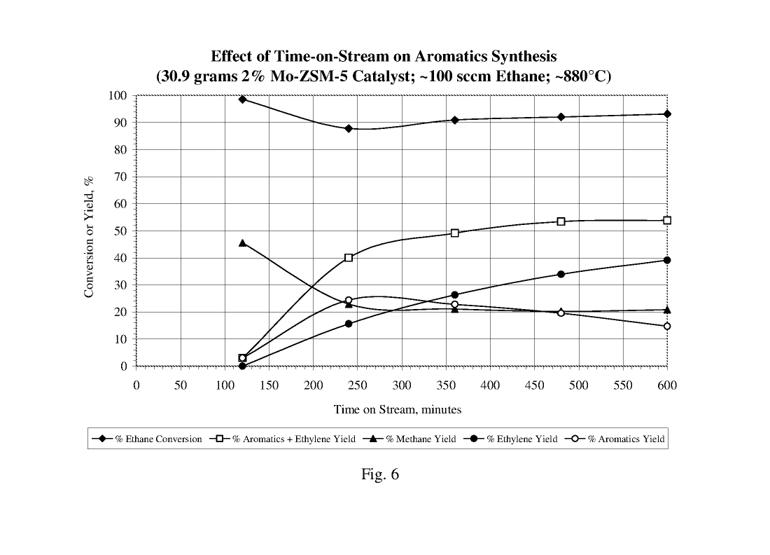

Results from additional experiments in the series showed that regardless of ethane feed rate, ethane conversion and ethylene yield increased sharply with temperature, Methane yield increased, but to a lower extent (from 2 to 3 percent at 752° C. to 9 to 15 percent at 874 to 885° C.). These results are summarized in FIG. 4.

The yield of methane decreased from 21 percent at the lowest ethane feed rate to 9 percent at the highest ethane feed rate. FIG. 5 summarizes the effects of ethane feed rate on ethane conversion and yields of aromatics, ethylene, and methane.

The effect of time-on-stream was determined from the results of Experiments 1 through 5 (98 to 106 sccm ethane feed rate at 880 to 891° C.). Results showed that after initial catalyst activation (Experiment 1; 99 percent ethane conversion), ethane conversion remained between 88 and 93 percent for time-on-stream from 120 to 600 minutes. The ethylene yield was zero during catalyst activation. However, ethylene yield showed a gradual increase from 16 to 39 percent for time-on-stream from 120 to 600 minutes. The measured yield of liquid aromatics was zero during catalyst activation, then increased to 24 percent at 240 minutes time-on-stream and gradually dropped to 15 percent by the end of the 600 minute time-on-stream test period. The combined aromatics plus ethylene yield gradually increased from 40 percent and reached at nearly steady value of about 53 to 54 percent after about 460 minutes. The methane yield was about 46 percent during catalyst activation, then became steady at about 20 percent for the duration of time-on-stream testing at 98 to 106 sccm ethane feed rate.

Except for Experiment 1 (catalyst conditioning) and Experiments 6 and 7 (lower temperature), the liquid aromatic product collected from Condenser 2a (which was the predominant product in all tests) contained a total of 74 to 88 percent by liquid volume of benzene plus toluene (based on GowMac 580 GC results externally calibrated to standard solutions). Further analysis by GC/MS showed additional aromatics in the form of naphthalene and methyl- and ethyl-forms of C6 and C7 aromatics. The concentration of benzene in all products from Experiments 2 through 5 and 8 through 10 was about 55 to 64 percent. The concentration of toluene increased from about 19 to 20 percent at lower ethane feed rates to as much as 32 percent at higher ethane feed rates.

In summary, the results of testing show the potential to achieve 70 percent or more conversion of ethane directly to liquid aromatics plus ethylene (a precursor to liquid aromatics) when operating at higher ethane feed rates and higher temperatures. The results also show minimum conversion of ethane to methane under the same range of conditions, leading to greater potential yield of more-valuable products.

### Experiment 11

The catalyst as described above was still producing significant amounts of aromatics plus ethylene through Experiment 10. However, literature suggests that similar catalysts can be expected to gradually degrade in performance over time. One likely reason for catalyst deactivation is deposition of carbon on catalyst surfaces and within catalyst pores, thereby blocking catalytic sites important for conversion of ethane to aromatics or ethylene.

The catalyst was subjected to an air oxidation regeneration step in order to establish whether the catalyst formulation used for testing described herein would remain active following regeneration and would therefore be suitable for longer-duration use.

The air oxidation experiment was conducted using the apparatus as shown in FIG. 3 with the catalyst remaining in place in the existing reactor. The system was first purged with helium gas at a flow of 50 to 100 sccm. The reactor was heated with flowing helium up to a temperature of about 350° C. The helium flow was stopped, and air from a compressed gas cylinder was started at a flow of about 155 sccm while the reactor continued to heat up. When the middle reactor temperature reached about 660° C., carbon monoxide and carbon dioxide in the exhaust gas rose sharply to about 1.8 and 15.3 percent by volume, respectively. Air flow was maintained at about 155 sccm while the reactor middle temperature was raised to about 785° C. over a period of about one hour. At this time, the carbon monoxide and carbon dioxide in the exhaust gas were 10.7 and 11.7 percent by volume, respectively, indicating significant consumption of oxygen in the inlet air. Conditions were held steady for another period of approximately 4.5 hours, during which the carbon monoxide concentration gradually decreased to about 5 percent while the carbon dioxide concentration increased to about 16 percent.

The regeneration test was then suspended by purging with helium and cooling prior to the next regeneration test segment. The second regeneration test segment was carried out in the same fashion as that described above except that air flow was doubled to about 310 sccm.

As the reactor middle temperature was increased to the 550° C. range, the carbon monoxide and carbon dioxide concentrations in the exhaust rose to about 1.8 and 5.2 percent, respectively. Upon further heating to a middle reactor temperature of about 600° C., the carbon monoxide and carbon dioxide concentrations remained at about 2 and 13 percent, respectively, over a period of about one hour. The carbon monoxide and carbon dioxide concentrations then gradually dropped to the 1 and 4 percent ranges, respectively, over the next one hour.

The reactor heater setting was increased to raise the reactor middle temperature to about 700° C. As the reactor heated, the carbon monoxide and carbon dioxide concentrations rose to as much as 6.5 and 11.3 percent, respectively, over a period of about 30 minutes. After the carbon monoxide and carbon dioxide concentrations dropped to about 0.2 and 0.6 percent, respectively, over a period of about one hour the reactor was cooled under a flow of helium gas.

Based on the exhaust gas flow rates and concentrations of carbon monoxide and carbon dioxide, about 11 grams of carbon were released from the catalyst by oxidation of the molybdenum carbide catalyst component (minor fraction) and carbon deposited during aromatization experiments. This represents an average of less than eight percent loss of carbon contained in the ethane feed as deposited carbon.

In summary, carbon was effectively removed from the 2% Mo ZSM-5 catalyst by controlled flow of air at temperatures between about 550 and 785° C.

### Experiment 12

After regenerating the 2% Mo ZSM-5 catalyst in air, a test was conducted under conditions similar to those of Experiment 10 above (ethane feed rate=482 sccm; middle reactor temperature about 813 to 849° C.). Within about 15 minutes, the exhaust gas composition became steady at about 35 percent hydrogen, 7 to 8 percent methane, 24 to 25 percent ethane, and 28 to 30 percent ethylene. The exhaust gas flow rate remained between 710 and 772 sccm.

Results near the conclusion of the two-hour experiment showed virtually no aromatics yield, but ethylene yield was about 52 percent, indicating that the catalyst had be reactivated. The ethylene yield was lower than the yield of about 70 percent achieved during Experiment 10. However, the middle reactor temperature during Experiment 12 averaged about 830° C. versus about 874° C. during Experiment 10, a substantially lower temperature considering the strong effect of temperature as illustrated in experiments 5 through 8. In addition, the ethane feed rate was somewhat higher (482 sccm versus 452 during Experiment 10).

In summary, regeneration in air removes carbon and allows for on-going use of the catalyst to produce ethylene and aromatics.

### Experiment 13

Experiment 13 was carried out to determine whether ethylene produced by conversion of ethane as described in Experiments 10 and 12 can be used as feed for synthesis of aromatics using the same 2% Mo ZSM-5 catalyst. For this experiment, the same apparatus shown in FIG. 3 including the same catalyst described above were used.

Test operating procedures as described above, including helium purge were carried out for Experiment 13, except ethylene gas from a compressed gas cylinder was fed to the system instead of ethane gas.

A flow of ethylene roughly comparable to that obtained in Experiments 10 and 12 (about 300 sccm ethylene) was fed to the reactor to represent the approximate flow rate of ethylene that would achieved using a two-step synthesis process as illustrated in FIG. 2.

A middle reactor temperature of about 500° C. was targeted for Experiment 13 based on scouting experiments and literature citations.

The exhaust gas flow rate gradually rose from about 51 sccm early in the experiment to an average of about 103 sccm during the final one hour of testing.

Ethylene conversion gradually dropped from 99 percent initially to about 83 percent over about 90 minutes and then decreased only slightly to about 77 percent over the next approximate 90 minute period.

The measured aromatics liquid product yield over the entire test duration (from Condenser 1, Condenser 2a, Condenser 2b, and Carbon Trap; see FIG. 3) was about 38 percent.

Most of the liquid product from Experiment 13 was collected from Condenser 1 (instead of Condenser 2a as in earlier single-step synthesis reactions in Experiments 1 through 10). The Condenser 1 product contained 3 to 5 percent benzene and 15 to 21 percent toluene based on GowMac 580 GC analysis. Additional analysis by GC/MS showed benzene and toluene along with significant amounts of C8, C9, and C10 aromatic compounds including xylene and naphthalene.

Operating conditions such as temperature and ethylene feed rate were not optimized during Experiment 13. Nevertheless, results showed substantial capabilities of the catalyst and reactor to produce high yields of aromatic compounds from ethane and ethylene.

In summary, ethylene (from catalytic decomposition of ethane or from ethylene produced by other conventional means) can be used as feed for the thermal catalytic synthesis of aromatic compounds using a ZSM based catalyst activated with molybdenum or other metals.

### REFERENCES

### Incorporated by Reference

1. Hagen, Anke and Frank Roessner, “Ethane to Aromatic Hydrocarbons: Past, Presnt, Future”, Catalyst Reviews: Science and Engineering, 42:4, 403-407, DOI: 10.1081/CR-100101952, 2000.
2. Qui, Ping, Jack H. Lunsford, and Michael P. Rosynek, “Characterization of Ga/ZSM-5 for the catalytic aromatization of dilute ethane streams”, Catalysis Letters, 52, 37-42, 1998.

What is claimed is:

1. A process for the synthesis of aromatic compounds including benzene, toluene, naphthalene, and other C8, C9, and C10 aromatic compounds by direct reaction of ethane-containing gas over a zeolite catalyst.

2. The method of claim 1 in which the ethane is present along with other alkanes and hydrocarbons recovered from natural gas wells.

3. The method of claim 1 in which the ethane is concentrated and recovered from natural gas produced from gas wells or as associated gas from oil production or from commercial sources as a feed stock for production of aromatic compounds.

4. The method of claim 1 in which the aromatics synthesis reaction is carried out at a temperature between 700 and 1000° C.

5. The method of claim 1 in which the process heat is supplied by combustion of hydrogen and other byproduct gases from ethane decomposition or by electrical heaters.

6. The method of claim 1 in which the aromatics synthesis reaction is carried out at a pressure between 0.1 and 10 bar absolute.

7. The method of claim 1 in which the aromatics synthesis reaction is carried out a gas hourly space velocity between 50 and 10000/hour.

8. The method of claim 1 in which the catalyst consists of a zeolite such as a H-ZSM-5 substrate to which an activating metal or metal oxide such as molybdenum is added at a concentration between 0.05 and 10 percent.

9. A process for the synthesis of aromatic compounds including benzene, toluene, naphthalene, and other C8, C9, and C10 aromatic compounds by a two-step reaction of ethane-containing gas over a zeolite catalyst or other suitable catalyst first to produce ethylene and hydrogen and next by reaction of the ethylene over a zeolite catalyst or other suitable catalyst to produce aromatic compounds and hydrogen.

10. The method of claim 9 in which the ethane is present along with other alkanes and hydrocarbons recovered from natural gas wells.

11. The method of claim 9 in which the ethane is concentrated and recovered from natural gas produced from gas wells or as associated gas from oil production or from commercial sources as a feed stock for production of aromatic compounds.

12. The method of claim 9 in which the ethane decomposition reaction is carried out at a temperature between 800 and 1000° C.

13. The method of claim 9 in which the process heat is supplied by combustion of hydrogen and other byproduct gases from ethane decomposition or by electrical heaters.

14. The method of claim 9 in which the ethane decomposition reaction is carried out at a pressure between 0.1 and 10 bar absolute.

15. The method of claim 9 in which the ethane decomposition reaction is carried out a gas hourly space velocity between 50 and 10000/hour.

16. The method of claim 9 in which the ethane decomposition catalyst consists of an H-ZSM-5 substrate to which an activating metal or metal oxide such as molybdenum is used at a concentration between 0.05 and 10 percent.

17. The method of claim 9 in which the ethylene is obtained from the first step of processing over a zeolite catalyst or other suitable catalyst or from conventional cracking of ethane, or from commercial sources as a feed stock for production of aromatic compounds.

18. The method of claim 9 in which aromatics synthesis from ethylene is carried is carried out at a temperature between 300 and 900° C.

19. The method of claim 9 in which aromatics synthesis from ethylene is carried out at a pressure between 0.1 and 10 bar absolute.

20. The method of claim 9 in which aromatics synthesis from ethylene is carried out a gas hourly space velocity between 50 and 10000/hour.

21. The method of claim 9 in which the aromatics synthesis from ethylene catalyst consists of an H-ZSM-5 substrate to which an activating metal or metal oxide such as molybdenum is used at a concentration between 0.05 and 10 percent.

22. The method of claim 9 in which a single reactor with different temperature zones are used for the two-step conversion of ethane to aromatics.

23. The method of claim 9 in which non-condensable byproduct gases are separated from any liquid aromatic products prior to feeding a second step reactor.

24. The method of claim 9 in which hydrogen is separated from any liquid aromatic products and ethane prior to feeding a second step reactor.

25. A device for the synthesis of aromatic compounds including benzene, toluene, naphthalene, and other C8, C9, and C10 aromatic compounds by direct reaction of ethane-containing gas over a zeolite catalyst.

26. The device of claim 25 in which the reactor comprises a fixed bed or a moving bed in which zeolite or other suitable catalyst is placed and periodically or continuously partially replaced with fresh catalyst.

27. The device of claim 25 in which the reactor is heated in whole or in part by external and/or internal electrical heaters.

28. The device of claim 25 in which the reactor is heated in whole or in part by external and/or internal indirect heat exchange passages fed in whole or in part by gases derived from combustion of byproduct gases (such as hydrogen) generated during aromatics synthesis or fuel gases (such as natural gas, ethane, or other gaseous fuels) obtained from commercial sources or solid and liquid fuels (such as oil, coal, or other solid and liquid fuels) obtained from commercial sources.

29. The device of claim 25 in which the reactor is heated in whole or in part by external and/or internal indirect heat exchange passages fed in whole or in part by gases derived from air oxidation of aromatics catalyst beds.

30. The device of claim 25 in which product gases from the first and/or second stage reactor are cooled in steps to sequentially recover compounds of higher melting temperature and lower vapor pressure first followed by compounds of lower melting temperature and higher vapor pressure by using condenser temperatures ranging from 100° C. to as low as −80° C.

31. The device of claim 25 in which parallel condensers are used in a mode that freezes aromatics compounds and in which one condenser is periodically heated to remove liquid product while the other is actively recovering solid product.

32. The device of claim 25 in which the final product gas from aromatics synthesis is passed through an activated carbon or other suitable bed to further remove aromatics compounds.

33. A device for the synthesis of aromatic compounds including benzene, toluene, naphthalene, and other C8, C9, and C10 aromatic compounds by a two-step reaction of ethane-containing gas over a zeolite catalyst first to produce ethylene and hydrogen and next by reaction of the ethylene over a zeolite catalyst to produce aromatic compounds and hydrogen.

34. The device of claim 33 in which the first stage and/or second stage reactor comprises a fixed bed or a moving bed in which zeolite or other suitable catalyst is placed and periodically or continuously partially replaced with fresh catalyst.

35. The device of claim 33 in which the reactor first stage and/or second stage reactor is heated in whole or in part by external and/or internal electrical heaters.

36. The device of claim 33 in which the first stage and/or second stage reactor is heated in whole or in part by external and/or internal indirect heat exchange passages fed in whole or in part by gases derived from combustion of byproduct gases (such as hydrogen) generated during aromatics synthesis or fuel gases (such as natural gas, ethane, or other gaseous fuels) obtained from commercial sources or solid and liquid fuels (such as oil, coal, or other solid and liquid fuels) obtained from commercial sources.

37. The device of claim 33 in which the first stage and/or second stage reactor is heated in whole or in part by external and/or internal indirect heat exchange passages fed in whole or in part by gases derived from air oxidation of aromatics catalyst beds.

38. The device of claim 33 in which hot exhaust gases from the first stage and/or second stage reactor indirectly exchange heat with colder first stage and/or second stage feed gases.

39. The device of claim 33 in which a single reactor is used to accommodate both reaction stages by utilizing external and/or internal thermal management devices such as heat exchangers to effectively create separate reaction zones.

40. The device of claim 33 in which product gases from the first and/or second stage reactor are cooled in steps to sequentially recover compounds of higher melting temperature and lower vapor pressure first followed by compounds of lower melting temperature and higher vapor pressure by using condenser temperatures ranging from 100° C. to as low as −80° C.

41. The device of claim 33 in which parallel condensers are used in a mode that freezes aromatics compounds and in which one condenser is periodically heated to remove liquid product while the other is actively recovering solid product.

42. The device of claim 33 in which the final product gas from aromatics synthesis is passed through an activated carbon or other suitable bed to further remove aromatics compounds.
